(19) United States
(12) Patent Application Publication
(10) Pub. No.: US 2021/0267462 A1
(43) Pub. Date: Sep. 2, 2021

(54) PATCH-TYPE THERMOMETER AND METHOD FOR MANUFACTURING SAME

(71) Applicant: AMOLIFESCIENCE CO., LTD., Seoul (KR)

(72) Inventor: Beom Jin KIM, Bucheon-si, Gyeonggi-do (KR)

(73) Assignee: AMOLIFESCIENCE CO., LTD., Seoul (KR)

(21) Appl. No.: 17/255,129

(22) PCT Filed: Jun. 26, 2019
(86) PCT No.: PCT/KR2019/007699

§ 371 Date: Dec. 22, 2020

(30) Foreign Application Priority Data
Jun. 27, 2018 (KR) 10-2018-0074196
Jun. 25, 2019 (KR) 10-2019-0075448

(51) Int. Cl. A61B 5/01; A61B 5/00

(52) U.S. Cl. CPC A61B 5/01; A61B 5/6833; A61B 5/0008; A61B 2562/0271; A61B 2562/166; A61B 2562/12; A61B 5/002

(57) ABSTRACT

A patch-type thermometer is provided. A patch-type thermometer according to an exemplary embodiment of the present invention comprises: a flexible circuit board which has an antenna pattern formed on at least one surface thereof and at least one driving chip mounted thereon; a temperature sensor mounted on the flexible circuit board so as to measure a user's body temperature; a heat transfer member electrically connected to the temperature sensor by means of a via hole and mounted on a surface opposite to a surface of the flexible circuit board on which the temperature sensor is mounted, so that the heat transfer member can come into direct contact with a user's skin; and a protective member surrounding the flexible circuit board so as to prevent the antenna pattern, the driving chip, and the temperature sensor from being exposed to the outside.

### CROSS REFERENCE TO RELATED APPLICATION

This application is a 35 U.S.C. 371 National Phase Entry Application from PCT/KR2019/007699, filed Jun. 26, 2019, which claims the benefit of Korean Patent Application Nos. 10-2018-0074196 filed on Jun. 27, 2018 and 10-2019-0075448 filed Jun. 25, 2019, the disclosures of which are incorporated herein in their entirety by reference.

### TECHNICAL FIELD

The present invention relates to a patch-type thermometer and a method of manufacturing the same.

### BACKGROUND

Generally, a body temperature is measured using a thermometer. The body temperature is accurately measured by the thermometer when a predetermined time has passed in a state in which the thermometer is worn on or is in contact with a body.

Accordingly, since another person should maintain a state in which the thermometer is in contact with a body of a measurement target person in order to measure a body temperature of a baby, a child, or a patient with reduced mobility, there is a problem of measuring the body temperature being cumbersome.

In addition, since the conventional thermometer is used in a method in which the thermometer is separated from the body of the target person and the body temperature is checked therefrom after being in contact with the body thereof for a predetermined time, there is an inconvenience in checking the temperature periodically or in real time.

### SUMMARY OF THE INVENTION

The present invention is directed to providing a patch-type thermometer that may be simply attached to a user's body and may simply check a user's body temperature in a state in which the patch-type thermometer is attached to a user's skin.

In addition, the present invention is also directed to providing a patch-type thermometer to which driving power is supplied using an energy harvesting method so that the patch-type thermometer may be formed in a thin film type.

In addition, the present invention is also directed to providing a method of manufacturing the patch-type thermometer allowing embedded electric components to be prevented from being damaged even when information such as a logo is printed thereon.

One aspect of the present invention provides a patch-type thermometer including a flexible circuit board provided with an antenna pattern formed on at least one surface thereof and at least one driving chip mounted thereon, a temperature sensor mounted on the flexible circuit board to measure a user's body temperature, a heat transfer member electrically connected to the temperature sensor through a via hole and mounted on a surface opposite to a surface, on which the temperature sensor is mounted, of the flexible circuit board to be in direct contact with the user's skin, and a protective member surrounding the flexible circuit board to prevent the antenna pattern, the driving chip, and the temperature sensor from being exposed to the outside.

In addition, the patch-type thermometer may be operated to transmit data measured by the temperature sensor to the outside when NFC tagging.

As an example, the antenna pattern may have a primary function of transmitting the data obtained through the temperature sensor when NFC tagging and a secondary function of receiving driving power needed by the driving chip through an energy harvesting method when NFC tagging. Accordingly, since a battery for driving the antenna pattern may be omitted, the flexibility of the patch-type thermometer may be improved, and the patch-type thermometer may be implemented in a thin film type.

In addition, the protective member may include an exposure hole formed to pass through a region therein corresponding to the heat transfer member, and the heat transfer member may be exposed to the outside through the exposure hole.

The heat transfer member may be formed to have a hemisphere or dome shape to be stably in contact with a user's skin.

The temperature sensor may be a digital-type temperature sensor. Accordingly, the temperature sensor may be completely buried inside the protective member so that airtightness thereof may be improved.

Another aspect of the present invention provides a method of manufacturing a patch-type thermometer, the method including preparing a flexible circuit board provided with an antenna pattern patterned thereon, a driving chip and a temperature sensor mounted thereon, and a heat transfer member mounted on a surface thereof opposite to a surface on which the temperature sensor is mounted, first forming an upper protective member formed of a flexible material to cover an upper surface of the flexible circuit board after the flexible circuit board is inserted into a first mold, and second forming a lower protective member formed of a flexible material to cove a lower surface of the flexible circuit board after the flexible circuit board integrated with the upper protective member is inserted into a second mold.

In addition, the method may further include printing information about at least one of a character, a number, and a figure on an exposure surface of the upper protective member after the first forming.

The second forming may further include forming an exposure hole in a region, which corresponds to the heat transfer member, in the lower protective member to expose at least a portion of the heat transfer member to the outside.

The second forming may further include forming a gel type bonding layer on one surface of the lower protective member.

### Advantageous Effects

According to the present invention, since a user's body temperature can be simply checked through near-field communication (NFC) tagging in a state in which a patch-type thermometer is attached to a skin, convenience in use can be improved.

In addition, in the present invention, since driving power needed by a driving chip is supplied using an energy harvesting method, a battery mounting space is omitted, and thus, the patch-type thermometer can be formed in a thin film type.

In addition, in the present invention, since damage to embedded electronic components can be prevented even when information such as a logo is printed thereon, a reliability of a product can be improved.

### BRIEF DESCRIPTION OF THE DRAWINGS

FIG. 1 is a view illustrating a patch-type thermometer according to one embodiment of the present invention.

FIG. 2 is a bottom view illustrating a state in which a release film is separated from that shown in FIG. 1.

FIG. 3 is a view illustrating an internal structure of FIG. 1.

FIG. 4 is a cross-sectional view taken along line A-A of FIG. 3.

FIG. 5 is a flowchart showing a method of manufacturing the patch-type thermometer according to one embodiment of the present invention.

FIGS. 6A-6E are a set of sequential views illustrating the method of manufacturing the patch-type thermometer according to one embodiment of the present invention.

### DETAILED DESCRIPTION

Hereinafter, embodiments of the present invention will be described in detail with reference to the accompanying drawings in order for those skilled in the art to easily perform the present invention. The present invention may be implemented in several different forms and is not limited to the embodiments described herein. Parts irrelevant to description are omitted in the drawings in order to clearly explain the present invention. In addition, components which are the same or similar to each other are assigned with the same reference numerals.

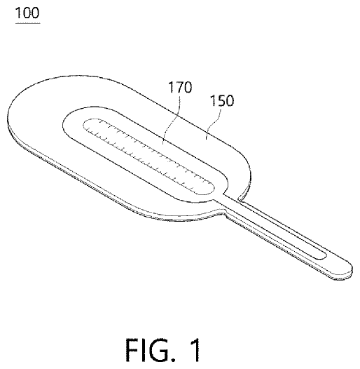

As illustrated in FIGS. 1 to 4, a patch-type thermometer 100 according to one embodiment of the present invention includes a flexible circuit board 110, an antenna pattern 120, a temperature sensor 130, a heat transfer member 140, and a protective member 150.

The flexible circuit board 110 may be a base on which various circuit elements are mounted or a circuit pattern for electrical connection is formed. As an example, the circuit elements may be a chipset configured to perform a predetermined function, and the circuit pattern may be an antenna pattern or wiring pattern for electrical connection.

Such a flexible circuit board 110 may be a known FPCB (flexible printed circuit board) having flexibility and formed of polyimide (PI), polyethylene terephthalate (PET), or the like.

In this case, the antenna pattern 120 may be formed on at least one surface of the flexible circuit board 110, and at least one driving chip 121 electrically connected to the antenna pattern 120 may be mounted on one surface of the flexible circuit board 110.

In addition, the temperature sensor 130 may be mounted on one surface of the flexible circuit board 110, and the temperature sensor 130 may be electrically connected to the driving chip 121 through a lead part 114.

As an example, the antenna pattern 120 may be a near-field communication (NFC) antenna for near-field communication, and the driving chip 121 may be an NFC driving chip configured to drive the antenna pattern 120.

Accordingly, the antenna pattern 120 may be driven by the driving chip 121 mounted on the flexible circuit board 110 and perform an emitter function of transmitting information obtained through the temperature sensor 130 to the outside using an NFC method.

Accordingly, data measured by the temperature sensor 130 may be transmitted to an external electronic device, for example, a mobile phone, through the antenna pattern 120 when NFC tagging.

Accordingly, a user simply checks data measured by the temperature sensor 130 through the mobile phone when NFC tagging.

In this case, when NFC tagging, the data transmitted to the mobile phone through the antenna pattern 120 may be displayed on the mobile phone through an application (APP) stored therein. In addition, the data transmitted to the mobile phone through the antenna pattern 120 may also be directly stored in the APP. Accordingly, the user may check a change trend of a temperature through the data stored in the APP.

In addition, the data transmitted to the mobile phone may also be transmitted to a separate server or another electronic device through a network connected to the mobile phone and may be stored in the server or the electronic device.

In the above description, the external electronic device is illustrated as the mobile phone but is not limited thereto and may also be a portable electronic device such as a tablet personal computer (PC) or a wearable device such as a smart watch.

Meanwhile, the antenna pattern 120 may have a primary function of transmitting information obtained through the temperature sensor 130 and a secondary function of receiving driving power needed by the driving chip 121. Here, the primary function may be a data transmission function, and the secondary function may be a wireless power receiving function.

That is, the antenna pattern 120 may receive power from an external device using an energy harvesting method and transmit the received power to the driving chip 121. Specifically, when NFC tagging, the antenna pattern 120 may wirelessly receive power from the external device to drive the driving chip 121.

Accordingly, when NFC tagging, the driving chip 121 may be driven using power received through the antenna pattern 120, and information obtained through the temperature sensor 130 may be transmitted to the external device through the antenna pattern 120.

Accordingly, since the patch-type thermometer 100 according to one embodiment of the present invention does not need a separate power source such as a battery for deriving the driving chip 121, a total weight thereof may be decreased, the separate power source such as the battery may be omitted, and thus the patch-type thermometer 100 can be implemented in an ultrathin film type.

The temperature sensor 130 may detect a user's body temperature to generate information about the user's body temperature. The temperature sensor 130 may be mounted on one surface of the flexible circuit board 110.

In this case, the temperature sensor 130 may be a digital-type temperature sensor and measure the user's body temperature based on heat transferred through the heat transfer member 140.

To this end, the temperature sensor 130 may be mounted on a first surface of the flexible circuit board 110, and the heat transfer member 140 may be mounted on a second surface of the flexible circuit board 110 to be in direct contact with a user's skin. In this case, the temperature sensor 130 and the heat transfer member 140 may be electrically connected through a via hole 112.

Here, the first surface and the second surface of the flexible circuit board 110 may be an opposite to each other. As an example, the first surface may be an upper surface of the flexible circuit board 110, and the second surface may be a lower surface of the flexible circuit board 110.

Accordingly, the temperature sensor 130 may not need to be exposed to the user's skin and may be mounted on the first surface, which is the same surface as a surface on which the driving chip 121 is mounted, of the flexible circuit board 110. In addition, since the temperature sensor 130 may be completely covered by the protective member 150, airtightness thereof can be improved.

In this case, the driving chip 121 may be disposed inside of the antenna pattern 120, the temperature sensor 130 may be disposed outside of the antenna pattern 120, and the driving chip 121 and the temperature sensor 130 may be electrically connected through the lead part 114 formed on at least one surface of the flexible circuit board 110.

In the present invention, the heat transfer member 140 may be mounted on the second surface of the flexible circuit board 110 and be in direct contact with the user's skin as described above. Accordingly, a body temperature transmitted from the user's skin may be transmitted to the temperature sensor 130 through the heat transfer member 140.

To this end, the heat transfer member 140 may be formed of a metal material having high conductivity.

Here, in a case in which the patch-type thermometer 100 according to one embodiment of the present invention is attached to the user's skin, the heat transfer member 140 may have a form capable of maintaining a state in which the heat transfer member 140 is continuously in contact with the user's skin. To this end, a central portion of the heat transfer member 140 may have a shape convexly protruding in one direction.

As an example, the heat transfer member 140 may be formed to have a hemisphere or dome shape.

Accordingly, in the case in which the patch-type thermometer 100 according to one embodiment of the present invention is attached to the user's skin, even when an attachment portion is a bent portion, the central portion of the heat transfer member 140 may maintain the state in which the central portion is continuously in contact with the user's skin. Accordingly, the heat transfer member 140 may smoothly transfer heat transferred from the user's skin to the temperature sensor 130.

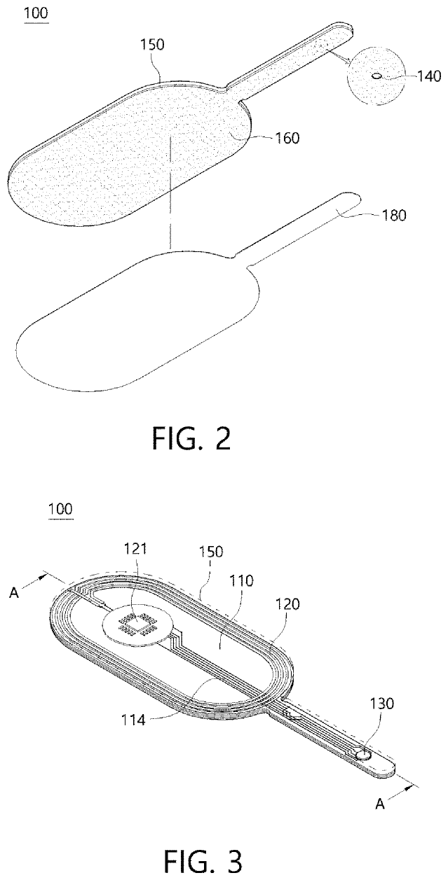

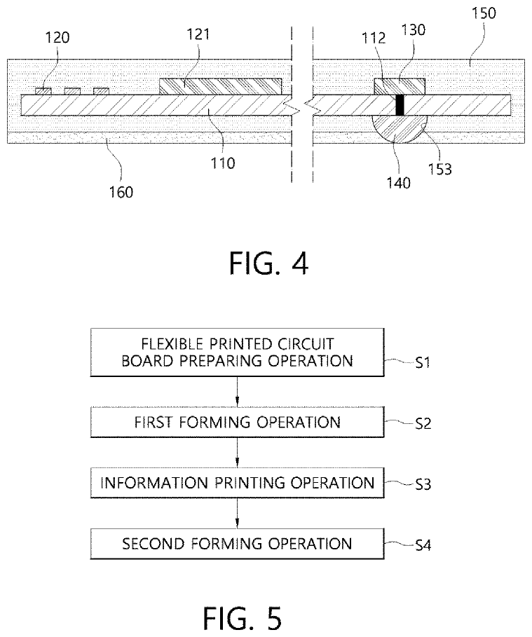

As illustrated in FIGS. 2 and 4, the heat transfer member 140 may be exposed to the outside through an exposure hole 153 formed in the protective member 150 which will be described below. Accordingly, in a case in which the patch-type thermometer 100 according to one embodiment of the present invention is attached to the user's body, the heat transfer member 140 may be continuously in direct contact with the user's skin.

Meanwhile, the patch-type thermometer 100 according to one embodiment of the present invention may include the protective member 150 surrounding the flexible circuit board 110 to prevent the flexible circuit board 110, the antenna pattern 120, the driving chip 121, and the temperature sensor 130 from being exposed to the outside.

In this case, as described above, the protective member 150 may include the exposure hole 153 formed in a region corresponding to the heat transfer member 140, and the heat transfer member 140 may be exposed to the outside through the exposure hole 153.

Accordingly, the protective member 150 may be formed to completely surround an entire portion of the patch-type thermometer 100 except for the heat transfer member 140.

That is, since the protective member 150 is formed to completely cover the upper and lower surfaces of the flexible circuit board 110, the protective member 150 may prevent the antenna pattern 120, the driving chip 121, the temperature sensor 130, and the flexible circuit board 110, excluding the heat transfer member 140, from being exposed to the outside.

In this case, the protective member 150 may be formed of a flexible material. Accordingly, even when the patch-type thermometer 100 according to one embodiment of the present invention is attached to a bent body part, since the patch-type thermometer 100 may be smoothly changed according to a bend of the user's body, adhesion therebetween can be improved.

As an example, the protective member 150 may be a molding part formed of an insulating resin material such as silicone. However, the protective member 150 is not limited thereto and may have a form formed of a fluoropolymer resin such as PET, polypropylene (PP), and polyethylene (PE) or release paper and the like, and any material having insulation and airtightness properties may be used for the protective member 150 without limitation.

Meanwhile, the patch-type thermometer 100 according to one embodiment of the present invention may include a bonding layer 160 formed on one surface of the protective member 150.

The bonding layer 160 may provide an adhesive force so that the patch-type thermometer 100 can be attached to the user's body. Here, the bonding layer 160 may be formed on a surface in which the exposure hole 153 for exposing the heat transfer member 140 to the outside is formed.

Accordingly, in a case in which the patch-type thermometer 100 is attached to the user's skin through the bonding layer 160, the heat transfer member 140 may be in direct contact with the user's skin.

As an example, the bonding layer 160 may be a gel type bonding layer and be formed of a material of which an adhesive force is restored when in contact with moisture. Accordingly, the bonding layer 160 may be repeatedly reusable.

However, the material of the bonding layer 160 is not limited thereto, and any known material capable of providing an adhesive force to the user's skin may be used for the bonding layer 160 without limitation.

In addition, the patch-type thermometer 100 according to one embodiment of the present invention may include an information display part 170 formed on one surface of the protective member 150. The information display part 170 may include information about one or more of characters, numbers, and figures.

As an example, the information display part 170 may be a logo or figure for aesthetics. Accordingly, the user may check various pieces of information through the information display part 170 to identify information about a product.

Meanwhile, the above-described patch-type thermometer 100 according to one embodiment of the present invention may be manufactured through the following method. In this case, the protective member 150 may be formed of a silicone material.

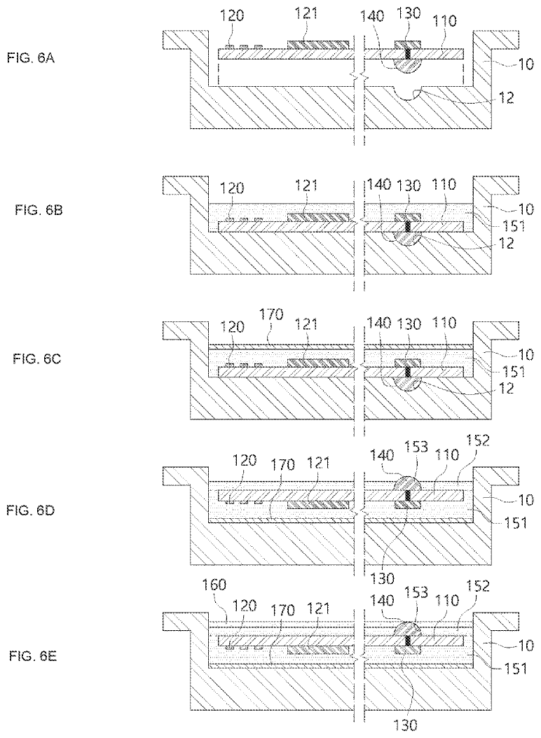

As an example, as illustrated in FIGS. 5 and 6A-6E, the method of manufacturing the patch-type thermometer according to one embodiment of the present invention may include a flexible circuit board preparing operation (S1), a first forming operation (S2), and a second forming operation (S4).

The flexible circuit board preparing operation (S1) is an operation of mounting or forming various electronic components such as the driving chip 121, the antenna pattern 120, the temperature sensor 130, and the heat transfer member 140 and a circuit pattern on the flexible circuit board 110.

That is, the driving chip 121, the temperature sensor 130, and the heat transfer member 140 may be mounted on both surfaces of the flexible circuit board 110, and the antenna pattern 120 may be formed on one surface of the flexible circuit board 110.

Here, since arrangement positions and mutual connection relationships among the driving chip 121, the temperature sensor 130, the heat transfer member 140, and the antenna pattern 120 disposed on the flexible circuit board 110 are the same as those of above-described contents, the detailed descriptions will be omitted.

In the method of manufacturing the patch-type thermometer according to one embodiment of the present invention, the first forming operation (S2) for forming an upper protective member 151 on the flexible circuit board 110 may be performed in the above-described state.

To this end, as illustrated in FIG. 6A, the flexible circuit board 110 may be inserted in a first mold 10, and as illustrated in FIG. 6B, in the state in which the flexible circuit board 110 is inserted into the first mold 10, a flexible material may fill the first mold 10. As an example, a molding material formed of a silicone material may fill the first mold 10 to cover the upper surface of the flexible circuit board 110.

Accordingly, since the upper protective member 151 formed of a flexible material is formed on the flexible circuit board 110, the antenna pattern 120, the temperature sensor 130, and the driving chip 121 mounted on the first surface of the flexible circuit board 110 may be protected by the upper protective member 151. Here, a bottom surface of the first mold 10 may have a shape corresponding to the lower surface of the flexible circuit board 110. As an example illustrated in FIG. 6A, a groove portion 12 formed to have a predetermined depth may be formed in the bottom surface of the first mold 10 at a position corresponding to the heat transfer member 140.

Accordingly, even when the heat transfer member 140 protrudes a predetermined height from the second surface of the flexible circuit board 110, the protruding height may be accommodated in the groove portion 12. Accordingly, even when an upper side of the flexible circuit board 110 is pressed in a state in which the lower surface of the flexible circuit board 110 is in close contact with the bottom surface of the first mold 10, the heat transfer member 140 can be prevented from being damaged or broken by an external force.

Meanwhile, the method of manufacturing the patch-type thermometer according to one embodiment of the present invention may include an information printing operation (S3) on an exposed surface of the upper protective member 151. As an example, the information printing operation (S3) may be performed after the first forming operation (S2). Accordingly, the information display part 170 including one or more among characters, numbers, and figures may be formed on the exposure surface of the upper protective member 151.

In this case, as illustrated in FIG. 6C, the information printing operation (S3) may be performed in a state in which the flexible circuit board 110 and the upper protective member 151 are seated in the first mold 10.

Accordingly, even when an external force is applied to the exposure surface of the upper protective member 151 during a process of printing the information display part 170 on the exposure surface of the upper protective member 151, since the heat transfer member 140 is protected by the groove portion 12 formed in the bottom surface of the first mold 10, the heat transfer member 140 can be prevented from being damaged or broken due to the external force as described above.

Then, in the method of manufacturing the patch-type thermometer according to one embodiment of the present invention, the second forming operation (S4), in which a lower protective member 152 covering the lower surface of the flexible circuit board 110 is formed, may be performed.

That is, in the case in which the method of manufacturing the patch-type thermometer according to one embodiment of the present invention includes the information printing operation (S3), the second forming operation (S4) may be performed after the information printing operation (S3) through a method similar to that of the above-described first forming operation (S2).

Specifically, after the information display part 170 is formed on the exposure surface of the upper protective member 151, the flexible circuit board 110 may be withdrawn from the first mold 10 and inserted into a second mold 20.

In this case, the flexible circuit board 110 may be inserted into the second mold 20 such that the upper protective member 151 is in close contact with a bottom surface of the second mold 20.

In this state, as illustrated in FIG. 6D, a flexible material may fill the second mold 20. As an example, a molding material formed of a silicone material may fill the second mold 20 to cover an exposure surface of the flexible circuit board 110.

Accordingly, since the lower protective member 152 formed of a flexible material is formed on one surface of the flexible circuit board 110, one surface of the flexible circuit board 110 can be prevented from being exposed to the outside.

In this case, the second forming operation (S4) may further include forming the exposure hole 153 in the lower protective member 152.

As an example, in the second forming operation (S4), the molding material may fill the second mold 20 so as not to cover at least a portion of the heat transfer member 140. Accordingly, the exposure hole 153 may be formed in the lower protective member 152 at a position corresponding to the heat transfer member 140, and the heat transfer member 140 may be exposed to the outside through the exposure hole 153.

Meanwhile, the method of manufacturing the patch-type thermometer according to one embodiment of the present invention may further include forming a gel type bonding layer on one surface of the lower protective member 152.

The forming of the gel type bonding layer may be performed after the second forming operation (S4), and the bonding layer 160 may be formed on one surface of the lower protective member 152.

As an example, as illustrated in FIG. 6E, the bonding layer 160 may be formed on an exposure surface of the lower protective member 152, and the bonding layer 160 may be attached to the user's skin.

In addition, a release film 180, which is removable, may also be additionally attached to an exposure surface of the bonding layer 160.

While the embodiments of the present invention have been described above, the spirit of the present invention is not limited to the embodiment proposed in this specification, and it will be understood by those skilled in the art that other embodiments may be easily suggested by adding, changing, and deleting components, and the other embodiments will fall within the spiritual range of the present invention.

1. A patch-type thermometer comprising:
a flexible circuit board provided with an antenna pattern formed on at least one surface thereof and at least one driving chip mounted thereon;
a temperature sensor mounted on the flexible circuit board to measure a user's body temperature;
a heat transfer member electrically connected to the temperature sensor through a via hole and mounted on a surface opposite to a surface, on which the temperature sensor is mounted, of the flexible circuit board to be in direct contact with the user's skin; and
a protective member surrounding the flexible circuit board to prevent the antenna pattern, the driving chip, and the temperature sensor from being exposed to the outside.

2. The patch-type thermometer of claim 1, which is operated to transmit data measured by the temperature sensor to the outside when NFC tagging.

3. The patch-type thermometer of claim 1, wherein the antenna pattern has a primary function of transmitting data obtained through the temperature sensor when NFC tagging and a secondary function of receiving driving power needed by the driving chip through an energy harvesting method when NFC tagging.

4. The patch-type thermometer of claim 1, wherein:
the protective member includes an exposure hole formed to pass through a region therein corresponding to the heat transfer member; and
the heat transfer member is exposed to the outside through the exposure hole.

5. The patch-type thermometer of claim 1, wherein the heat transfer member is formed of a metal material.

6. The patch-type thermometer of claim 1, wherein the heat transfer member is formed to have a hemisphere or dome shape.

7. The patch-type thermometer of claim 1, wherein the protective member is formed of a flexible material.

8. The patch-type thermometer of claim 1, wherein the temperature sensor includes a digital-type temperature sensor.

9. The patch-type thermometer of claim 1, wherein a gel type bonding layer is formed on one surface of the protective member.

10. The patch-type thermometer of claim 1, wherein:
the driving chip is disposed inside of the antenna pattern;
the temperature sensor is disposed outside of the antenna pattern; and
the driving chip and the temperature sensor are electrically connected through a lead part patterned on at least one surface of the flexible circuit board.

11. A method of manufacturing a patch-type thermometer, comprising;
preparing a flexible circuit board provided with an antenna pattern patterned thereon, a driving chip and a temperature sensor mounted thereon, and a heat transfer member mounted on a surface thereof opposite to a surface on which the temperature sensor is mounted;
first forming an upper protective member formed of a flexible material to cover an upper surface of the flexible circuit board after the flexible circuit board is inserted into a first mold; and
second forming a lower protective member formed of a flexible material to cover a lower surface of the flexible circuit board after the flexible circuit board integrated with the upper protective member is inserted into a second mold.

12. The method of claim 11, further comprising printing information about at least one of a character, a number, and a figure on an exposure surface of the upper protective member after the first forming.

13. The method of claim 11, wherein the second forming further includes forming an exposure hole in a region, which corresponds to the heat transfer member, in the lower protective member to expose at least a portion of the heat transfer member to the outside.

14. The method of claim 11, wherein the second forming further includes forming a gel type bonding layer on one surface of the lower protective member.
